(19) United States
(12) Patent Application Publication
(10) Pub. No.: US 2005/0004216 A1
(43) Pub. Date: Jan. 6, 2005

(54) Treating asthma, chronic obstructive pulmonary disease and/or other respiratory difficulties

(76) Inventors: Dianne Cadwallader, Auckland (NZ); Timothy David Meakin, Auckland (NZ); Craig Leonard Heatley, Auckland (NZ)

Correspondence Address: JACOBSON HOLMAN PLLC, 400 SEVENTH STREET N.W., SUITE 600, WASHINGTON, DC, 20004, US

(21) Appl. No.: 10/490,864

(22) PCT Filed: Sep. 27, 2002
(86) PCT No.: PCT/NZ02/00195

(30) Foreign Application Priority Data
Sep. 28, 2001 (NZ) 514536

(51) Int. Cl. A61K 31/23

(52) U.S. Cl. 514/552

(57) ABSTRACT

A method of treatment and/or prophylaxis of a mammal for at least the symptoms of treating asthma, chronic obstructive pulmonary disease and/or other respiratory difficulties which comprises or includes administering or having self administered to such mammal an effective amount of either (a) cetyl myristate, or (b) cetyl myristate and cetyl palmitate.

### TECHNICAL FIELD

The present invention relates to a method of treatment and/or prophylaxis of at least the symptoms of asthma, chronic obstructive pulmonary disease and/or other respiratory difficulties.

### BACKGROUND

Asthma is a condition that affects your airways primarily the small tubes that carry air in and out of the lungs. Those who suffer Asthma have airways that are almost always red and sensitive. The redness usually indicates that the airways are inflamed. There are various Asthma triggers and can include things such as a cold or flu, exercise, allergies to things such as pollen, fur or dust mites. Essentially Asthma causes breathing problems, it can he life threatening and is a disease that affects the lungs. Chronic obstructive pulmonary disease is an extreme example of respiratory disease and currently is not known to have a cure.

Asthma can have very damaging effects on a persons normal way of life where they may no longer exercise or get out and about to enjoy themselves for fear of having an Asthma attack. To control this outset of an Asthma attack people take various prescribed medication including FLIXOTIDE™, RESPICORT™ etc or simply do not bring themselves into a situation in which an Asthma attack could be brought about.

The present invention has surprisingly determined that the administration (particularly by ingestion) of cetyl myristate, and particularly cetyl myristate in conjunction with cetyl palmitate, provides an effective treatment of at least the symptoms of asthma, chronic obstructive pulmonary disease and/or other respiratory difficulties. This effect is experienced in as little as 2 weeks for those patients suffering chronic obstructive pulmonary disease.

Cetyl myristate and cetyl palpitate can each be sourced from animals or vegetables. Cetyl myristate is not to be mistaken for cetyl myristoleate which is also a fatty acid derived traditionally from spermaceti by saponification and more recently from the tallow of bovine(s).

Reference is made to U.S. Pat. No. 4,113,881 where it is disclosed that the administration of an effective amount of cetyl myristoleate to a mammal is useful in inhibiting or relieving the symptoms of inflammatory rheumatoid arthritis in mammals. Also in U.S. Pat. No. 5,569,676 there is disclosure of the use of cetyl myristoleate in the treatment of osteo-arthritis.

It is thought that cetyl myristate has a negligible anti-arthritic activity in laboratory experiments and reference is made to the website www.gcinutrients.com/Newletter.com. However this point is arguable and a product known as cetyl myristate sold by Amerex Corporation of 770 Sycamore Avenue, Suite J148, Vista, Calif. 92083, USA purports that cetyl myristate is useful for the treatment of arthritis.

Cetyl myristate is derived from the saturated fatty acid, myristic acid. This acid is found in nutmeg butter, in the fats of Myristicaceae, in palm seed fats, milk fats and also sperm whale oil. Reference is made to U.S. Pat. No. 2,481,365 which discloses the preparation of mystic acid from tall-oil fatty acids. It is to be noted that Amerex Corporation source the cetyl myristate used in their products from sunflower oil. See their website at www.hollinet.com.

Cetyl palmitate is derived from the fatty acid, palmitic acid which occurs as the glycerol ester in many oils and fats such as palm oil or Chinese vegetable tallow. A synthetic method of preparation is to react palmitoyl chloride and cetyl alcohol in the presence of magnesium. See the Merck Index, 12th edition at page 336. Reference is also made to U.S. Pat. No. 3,169,099 which discloses a biosynthetic method of producing cetyl palmitate.

It is an objection of the present invention to provide a medicament to aid in the treatment of asthma, chronic obstructive pulmonary disease and/or other respiratory difficulties which will provide an alternative to existing treatments or to provide the public with a useful choice.

### DISCLOSURE OF INVENTION

As indicated earlier the present invention is directed to the, treatment and/or prophylaxis of at least the symptoms of asthma, chronic obstructive pulmonary disease and/or other respiratory difficulties reliant upon administration (whether by self administration or otherwise) of either cetyl myristate or cetyl myristate and cetyl palmitate (whether given simultaneously in admixture or not or given serially or co-administration).

The present invention also encompasses the prospect of dosage forms that in some instances might contain cetyl myristate alone and in other instances both cetyl myristate and cetyl palmitate and dosage regimes that might use one dosage form or both.

Without being bound to our theory we believe that the present invention has a beneficial effect on Mast cells by stabilising these cells. Mast cells have Immunoglobulin receptors on their surfaces and are known to mediate aspects of allergic and inflammatory reactions. See Renew of Medical Physiology, by William F Garnong [15 ED].

It is believed that the stabilisation of these Mast cells prevents the allergic and inflammatory reaction occurring in the body that is responsible for asthma, chronic obstructive pulmonary disease and/or other respiratory difficulties. It is also thought this theory is equally applicable to our other patent applications as described in our New Zealand Patent Application No's. 504524, 504525/507228, 504526 and 502779, and also as described in our corresponding PCT Applications; PCT/NZ01/00084, PCT/NZ01/00085 and PCT/NZ01/00086.

### STATEMENTS OF INVENTION

In a first aspect the invention is a method of treatment and/or prophylaxis of a mammal for at least the symptoms of treating asthma, chronic obstructive pulmonary disease and/or other respiratory difficulties which comprises or includes administering or having self administered to such mammal an effective amount of either
(a) cetyl myristate, or
(b) cetyl myristate and cetyl palmitate.

Preferably and administration is orally of (b) whether as a mixture of both cetyl myristate and cetyl palmitate, or serially.

Preferably the effective amount is of (b).

Preferably said administration is with a mixture of cetyl myristate in conjunction with cetyl palmitate where the cetyl myristate comprises from 50 to 98% w/w of the mixture.

Preferably said effective amount of (a) or (b) is by means of one or more capsules.

The method also extends to related conditions, eg; accelerated wound healing where a composition as disclosed in U.S. Pat. No. 4,775,291 can at least sometimes be supplemented by use of the present invention methodology.

In another aspect the invention is an oral pharmaceutical composition for treating asthma, chronic obstructive pulmonary disease and/or other respiratory difficulties which comprises or includes both cetyl myristate and cetyl palmitate.

Preferably said cetyl myristate comprises at least 50% w/w of the composition.

Preferably said composition also includes at least one pharmaceutically acceptable excipient and/or diluent.

In still another aspect the invention is an oral dosage unit effective in the treatment of asthma, chronic obstructive pulmonary disease and/or other respiratory difficulties, said dosage unit having either
(a) cetyl myristate, or
(b) a mixture of cetyl myristate and cetyl palmitate.

Preferably said dosage unit is (b) and said cetyl myristate in any such mixture comprises from 50 to 98% w/w of the mixture.

In another variant the dosage unit has (a) only and there is between 5 to 400 mg of cetyl myristate.

Preferably in the dosage use, where (b) is present, there is from 5 to 400 mg of the mixture of cetyl myristate and cetyl palmitate.

Preferably (a) or (b) is in a capsule.

Preferably said capsule also includes a pharmaceutically acceptable excipient and/or diluent.

Preferably the dosage unit includes silicon dioxide.

Preferably the dosage unit also contains calcium phosphate and/or magnesium oxide.

Preferably the dosage unit also includes additionally at least one trace element.

In another aspect the invention is a liquid dosage unit being also an oral dosage unit as aforesaid.

In another aspect the invention is tile use, in the manufacture of oral dosage units for the treatment or prophylaxis of at least the symptoms of asthma, chronic obstructive pulmonary disease and/or other respiratory difficulties in a mammal, of
(a) cetyl myristate, or
(b) a mixture of cetyl myristate and cetyl palmitate, or
(c) cetyl palmitate.

In another aspect the invention is the use, in the manufacture of oral dosage Its for the treatment of asthma, chronic obstructive pulmonary disease and/or other respiratory difficulties or prophylaxis of at least the symptoms of asthma, chronic obstructive pulmonary disease and/or other respiratory difficulties in a mammal, or
(i) cetyl myristate and
(ii) cetyl palmitate.

The mixture can use cetyl myristate available from a commercial source such as EHP Products Inc., PO Box 20727, Mt Pleasant S.C. 29465 or at Amerex Corporation, 770 Sycamore Avenue Suite J148 Vista, Calif. 92083.

The mixture can use cetyl palmitate derived from a source such as, for example, Quimica Croda, S A de C. V, Circuito Médicos No.47. Apdo. Postal 71-A Cd. Satélite, 53100 Naucalpan, Edo. de México, México or online at wvw.butterburandsage.com.

Most ideally however the mixture is synthetised from starting materials utilizing the procedures as disclosed in New Zealand Patent Specification No. 332959 which involves reacting both myristic acid and palmitic acid with a cetyl alcohol At an elevated temperature in the presence of at least one acid catalyst and at least one aromatic hydrocarbon. The aromatic hydrocarbon fraction then contains the cetyl myristate and cetyl palmitate from whence it can be crystallised.

The full content of NZ 332959 is here incorporated by way of reference.

This crystallised form can then be ground up, dissolved and mixed with a suitable general pharmacy liquid to be administered to a person. The crystals are usually dissolved in hot water before adding to the pharmacy liquid which is usually a sugar syrup available from most pharmaceutical companies. The liquid is made up to a concentration of 70% w/v.

Alternatively the crystals may be ground up into a powder and combined with magnesium oxide, silicon oxide and fine di-calcium phosphate. This powder can then be transferred into capsules for oral ingestion into the body. The capsules used are VEGICAP™ that are non-gelatin containing.

The mode of administration is preferably oral. The dosage unit can be either a swallowable capsule or some alternative (preferably having the active ingredient(s) as a wax-like solid or can be an orally consumable liquid composition (eg; made up with a general pharmacy type carrier such as methyl cellulose)).

Other modes of administration can include transdermal, sublingual, parenteral, and suppository delivery.

The oral administration for the treatment of asthma, chronic obstructive pulmonary disease and/or other respiratory difficulties can be in addition to any other medicament administered for such ailment whether administered orally, topically, parenterally, sublingually, etc.

In practice the prevent invention will involve ideally oral self administration of effective quantities of cetyl myristate alone or more preferably as a mixture of both cetyl myristate and cetyl palmitate.

Preferably in any such mixture the cetyl myristate comprises at least about half of the mixture or the serial application on a weight to weight basis. It is envisaged that daily doses will vary depending on patient needs and may rage from 1 to 20 capsules per day. A capsule ideally contains between 5 to 370 mg of the mixture or cetyl myristate.

Trials with a variety of patients reliant upon dosage forms of cetyl myristate alone have shown favourable responses insofar as relief from the symptoms of asthma, chronic obstructive pulmonary disease and/or other respiratory difficulties is concerned. It has been found however that enhanced benefits occur where there is at least a small proportion of cetyl palmitate in addition to the cetyl myristate and it is to the use of one such ratio of these active ingredients that the following trial examples relate.

Examples of use follows. Each briefly describes tic patient's condition before and after the stated treatment using dosage forms (ie; “of the invention”) each having about 350 mg of the mixture of cetyl myristate and cetyl palmitate. That mixture comprises by weight 95% cetyl myristate and 5% cetyl palmitate by weight manufactured by the process as disclosed in NZ Patent Specification No. 332959. In addition added excipients were present in the admixture and then encapsulated in the non gelatin two part capsule case.

Accordingly the present invention consists in a method of treatment for asthma, chronic obstructive pulmonary disease and/or other respiratory difficulties (or other mammal) which comprises administering or having self administered to such human or other mammal an effective amount of either
(a) cetyl myristate, or
(b) cetyl myristate and cetyl palmitate.

Preferably said administration and/or self administration is by ingestion.

Preferably the administration and/or self administration is with a mixture of cetyl myristate in conjunction with cetyl palmitate where the cetyl myristate comprises, from 50 to 98% w/w.

In a further aspect the present invention consists in the use of an effective amount of either
(a) cetyl myristate, or
(b) cetyl myristate and cetyl palmitate
   in the manufacture of a dosage unit or pharmaceutical composition for oral ingestion useful in the treatment of asthma, chronic obstructive pulmonary disease and/or other respiratory difficulties.

Preferably said use involves the use of an appropriate encompassing capsule.

The present invention also consists in a pharmaceutical composition for treating asthma, chronic obstructive pulmonary disease and/or other respiratory difficulties which comprises an effective amount of cetyl myristate with an effective amount of cetyl palmitate.

In a further aspect the present invention consists in a dosage unit effective in the treatment of asthma, chronic obstructive pulmonary disease and/or other respiratory difficulties said dosage unit comprising either
(a) cetyl myristate in an appropriate orally deliverable dosage unit, or
(b) a mixture of cetyl myristate and cetyl palmitate in a suitable orally administrable dosage unit.

Preferably said cetyl myristate in any such mixture comprises from 50 to 98% w/w of the mixture.

In still a further aspect the present invention consists in a dosage unit In the form of a capsule for treating asthma, chronic obstructive pulmonary disease and/or other respiratory difficulties capable of releasing its content once ingested orally, said contents being a mixture of cetyl myristate with cetyl palmitate.

Preferably said cetyl myristate comprises from 50 to 98% w/w of the mixture

Preferably said contents is a wax like powder.

Preferably said powder is placed inside a capsule eg. a gelatine capsule without an end.

Preferably said capsule may include a pharmaceutically acceptable excipient.

Preferably said pharmaceutically acceptable excipient is in solid form.

Preferably said pharmaceutically acceptable excipients includes trace elements such as calcium phosphate or magnesium oxide.

In still a further aspect the present invention consists in a liquid or other soluble form which comprises, a mixture of
(a) cetyl myristate, or
(b) a mixture of cetyl myristate and cetyl palmitate where the mixture maybe carried in a suitable liquid for oral ingestion useful in the treatment of asthma, chronic obstructive pulmonary disease and/or other respiratory difficulties.

Preferably said suitable liquid is a general pharmacy liquid.

Preferably said cetyl myristate and any such mixture comprises from 50-98% w/w of the mixture.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

The invention consists in the foregoing and also envisages constructions of which the following gives example.

### TRIAL EXAMPLES

Patient 1 is Male and is 50 Years of Age.

Patient 1 has suffered from chronic obstructive pulmonary disease for the past 2 years. Patient 1 was previously on prescribed medication including 1 canister of VENTOLIN™ as required, at a rate of one canister per week (each canister has at least 200 doses). Patient 1 was also prescribed FLIXOTIDE™ at a rate of 200 micrograms twice daily and BAMBEC™ at a rate of 10 milligrams per day.

At the first appointment Patient 1 was provided with capsules of a dosage unit as described in invention for a dosage regime of 4 capsules, three times daily.

Within 2 weeks Patient 1's health began to improve with breathing becoming easier.

Patient 1 is now on a dosage regime of 2 capsules, twice daily and now only uses one canister of VENTOLIN™ every two weeks and the amount of FLIXOTIDE™ has also significantly reduced.

Patient 2 is Male and is 74 Years of Age.

Patient 2 suffers chronic obstructive pulmonary disease and has been an asthmatic for many years.

Patient 2 was previously on prescribed medication including FLIXOTIDE™ 1 putt twice daily, NUELIN™ tablets 1 350 mgs tablet twice daily, INTAL™ dose twice daily and RESPOLIN™.

At the first appointment Patient 2 was provided with capsules of a dosage unit as described in this invention for a dosage regime of four capsules, four times daily.

Patient 2 after 2 months, no longer needed to use RESPOLIN™ and his other prescribed medications were significantly reduced. Patient 2 is now on maintenance dose of 2 capsules twice daily.

Patient 3 is Male and is 59 Years of Age.

Patient 3 is an asthmatic. His previously prescribed medication included 1 atomiser canister of RESPOLIN™, where he was taking 8 puffs daily. This canister lasted one mouth.

At the first appointment Patient 3 was provided with capsules of a dosage unit as described in this invention for a dosage regime of 4 capsules three times daily which was also taken in conjunction with 1 capsule (125 micrograms) of FLIXOTIDE™ daily.

After taking the present invention for 2 months Patient 3 no longer needed RESPOLIN™ and now continues to do well on a maintenance dose of 2 capsules daily.

Patient 4 is Female and is 25 Years of Age.

Patient 4 suffers chronic asthma, is unable to exercise and was often hospitalised for asthma related incidences.

Her previous prescribed medication included 1 canister of VENTOLIN™ being used at a rate of 1-2 puffs every 4 hours, this canister would last a week, FLIXOTIDE™ and numerous courses of oral prednisone.

At the first appointment Patient 4 was provided with capsules of a dosage unit as described in this invention for a dosage regime of 4 capsules, four time daily.

Patient 4 has been on this dosage rate for the past 12 months and is now using only one VENTOLIN™ canister that lasts 3-4 months

Patient 4 is now able to exercise and living a normal life.

Patient 4 is now on a maintenance dose of 3 capsules twice daily.

Patient 5 is Female and is 82 Years of Age.

Patient 5 is asthmatic and suffers from chronic obstructive pulmonary disease. Her previously prescribed medication included ATROVENT FORTE™ at a rate of 4 puffs daily or as required. NUELIN SR™ at a rate of 250 milligrams twice daily, and FLIXOTIDE™ of 550 micrograms twice daily. Patient 5 has taken this medication for a number of years.

At the time of treatment her Peak Flow (a measurement of lung capacity) was 106 and she had a very heavy chest.

At the first appointment Patient 5 was provided with capsules of a dosage unit as described in this invention for a dosage regime of 4 capsules twice daily.

After being on this dosage regime for the past year, her chest has now cleared and her Peak Flow has now increased to 150. She now visits the doctor once every few month as opposed to nearly every month for her respiratory problems.

Patient 5 now has maintained her Peak Flow rate at 150 and has reduced the amount of NUELIN™ and ATROVENT FORTE™. She continues to do well on a maintenance dose of 3 capsules twice daily.

Patient 6 is Female and is 59 Years of Age.

Patient 6 has suffered asthma since 1986 when she was diagnosed but has always had breathing difficulties before this date and believes she was not diagnosed for many years. Patient 6 has been hospitalised twice for acute asthma attacks and was on prescribed medication including VENTOLIN™ at a rate of 3 or 4 puffs daily plus BECOTIDE™ at a rate of 2 puffs twice daily.

At her first appointment Patient 6 was provided with capsules of a dosage unit as described in this invention for a dosage regime of 4 capsules four time daily.

Since taking the invention Patient 6 has not been hospitalised and her VENTOLIN™ intake is now reduced to only 1 or 2 puffs every 2-3 months and when she feels a cold or infection developing she will use BECOTIDE™ once a day, otherwise she has ceased all other prescribed medication.

This Patient now continues to do well on a maintenance dose of 4 capsules twice daily in the morning and evening.

Patient 7 is Female and is 7 Years of Age.

Patient 7 is an asthmatic. She has always had a wheezy cough and was constantly sick with Bronchitis. Her prescribed medication included BECOTIDE™ inhaled steroids and FLIXOTIDE™. At the age of 4 her medication also included FLIXOTIDE™ at a rate of 1 puff of 25 micrograms twice daily.

At the first appointment Patient 7 was provided with capsules of a dosage rate as described in this invention for a dosage regime of 2 capsules, three times daily.

Patient 7 has been on Meracol for the past two years and now no longer uses any prescribed medication except in the winter months when her mother thinks that she is starting to get a cold. Her mother will then give her FLIXOTIDE™.

Patient 7 now continues to do well on a maintenance dose of one capsule twice daily.

Patient 8 is Male and is 4 Years of Age.

Patient 8 at 4 months of age had been prescribed oral VENTOLIN™ and BECOTIDE JUNIOR™ for his wheezy cough and Bronchitis and FLIXOTIDE™ at 1 puff of 25 micrograms twice daily. At the age of 8 months Patient 8 was nebulised. Patient 8 had constant ear and nose infections.

At the first appointment Patient 8 was provided with capsules of a dosage unit as described in this invention for a dosage regime of 1½ capsules, three times daily.

After one week the wheeziness stopped. He has now been on this dosage rate for the past 17 months and is no longer taking VENTOLIN™ or FLIXOTIDE™.

Visits to the Doctor for respiratory related illnesses have been zero over the past year. Whereas before he was visiting the Doctor every 2 weeks.

Patient 8 now continues to do well on a maintenance of 1-2 capsules daily.

1-24. (canceled).

25. A method of treatment and/or prophylaxis of at least the symptoms of asthma, chronic obstructive pulmonary disease and/or other respiratory difficulties in a mammal, which comprises or includes administering or having self administered to such mammal effective amounts of cetyl myristate and cetyl palmitate.

26. A method as claimed in claim 25 wherein said administration is orally of a mixture of both cetyl myristate and cetyl palmitate, or serially.

27. A method as claimed in claim 26 wherein said administration is with a mixture of cetyl myristate in conjunction with cetyl palmitate where the cetyl myristate comprises from 50 to 98% w/w of the mixture.

28. The method as claimed in claim 27 wherein said administration is by means of one or more capsules.

29. A method as claimed in claim 28 wherein both cetyl myristate and cetyl palmitate and/or an admixture are administered in the ratio w/w of 95:5 respectively.
